(19) United States
(12) Patent Application Publication
(10) Pub. No.: US 2024/0423613 A1
(43) Pub. Date: Dec. 26, 2024

(54) SYSTEMS AND METHODS FOR TUNING ABSORPTION PATHWAYS IN BIOABSORBABLE MATERIALS

(71) Applicant: Cilag GmbH International, Zug (CH)

(72) Inventors: BRODY FROST, Fairfield, OH (US); Madelaine Franzoni, Cincinnati, OH (US); Michael J. Vendely, Lebanon, OH (US); Peyton Hopson, Cincinnati, OH (US)

(21) Appl. No.: 18/485,083

(22) Filed: Oct. 11, 2023

Related U.S. Application Data
(60) Provisional application No. 63/522,660, filed on Jun. 22, 2023.

(51) Int. Cl. A61B 17/064; A61L 17/00; A61L 17/10; A61L 17/14

(52) U.S. Cl. CPC A61B 17/0644; A61L 17/005; A61L 17/10; A61L 17/145; A61B 2017/00004; A61L 2300/604

(57) ABSTRACT

The disclosed technology includes a bioabsorbable material configured to be delivered to tissue. The material includes a porous body comprising at least one polymer comprising a first zone comprising a first crosslink density and a second zone comprising a second crosslink density different than the first zone. The first and second zones form a gradient of compression strength along a portion of the porous body.

### CROSS-REFERENCE TO RELATED APPLICATIONS

This application claims priority under 35 U.S.C. § 119 to U.S. Provisional Patent Application No. 63/522,660, filed Jun. 22, 2023, the entire contents of which are fully incorporated herein by reference.

### FIELD

The present invention relates generally to systems and methods for tuning absorption pathways in bioabsorbable materials via chemical backbones and/or coatings.

### BACKGROUND

Surgical staplers are used in surgical procedures to close openings in tissue, blood vessels, ducts, shunts, or other objects or body parts involved in the particular procedure. The openings can be naturally occurring, such as passageways in blood vessels or an internal organ like the stomach, or they can be formed by the surgeon during a surgical procedure, such as by puncturing tissue or blood vessels to form a bypass or an anastomosis, or by cutting tissue during a stapling procedure.

Most staplers have a handle (some of which are directly user operable, others of which are operable by a user via a robotic interface) with an elongate shaft extending from the handle and having a pair of movable opposed jaws formed on an end thereof for holding and forming staples therebetween. The staples are typically contained in a staple cartridge, which can house multiple rows of staples and is often disposed in one of the two jaws for ejection of the staples to the surgical site. In use, the jaws are positioned so that the object to be stapled is disposed between the jaws, and staples are ejected and formed when the jaws are closed, and the device is actuated. Some staplers include a knife configured to travel between rows of staples in the staple cartridge to longitudinally cut and/or open the stapled tissue between the stapled rows.

### SUMMARY

There is provided, in accordance with an example of the present invention, a bioabsorbable material configured to be delivered to tissue. The material can include a first polymerizable compound and a second polymerizable compound. The first polymerizable compound can include a reaction product of a polyol and an isocyanate and can be configured to degrade according to a first degradation profile. The second polymerizable compound can be configured to degrade according to a second degradation profile different than the first degradation profile. The material can undergo a phase transition in response to exposure to a fluid comprising at least one of a predetermined temperature, an enzyme-catalyst, and a predetermined pH.

There is provided, in accordance with an example of the present invention, a bioabsorbable material configured to be delivered to tissue. The material can include a copolymer backbone. The copolymer backbone can include a first polymerizable compound and a second polymerizable compound. The first polymerizable compound can include a reaction product of a polyol and an isocyanate and can be configured to degrade according to a first degradation profile. The second polymerizable compound can be configured to degrade according to a second degradation profile different than the first degradation profile. The material can undergo a phase transition in response to exposure to the fluid comprising a predetermined temperature.

There is provided, in accordance with an example of the present invention, a bioabsorbable material configured to be delivered to tissue. The material can include a first polymerizable compound and a second polymerizable compound. The first polymerizable compound can include a reaction product of a polyol and an isocyanate and can be configured to degrade according to a first degradation profile. The second polymerizable compound can coat the first polymerizable compound such that the coating can prevent the first polymerizable compound from exposure to a fluid ingress for a predetermined period of time. The material can undergo a phase transition in response to exposure to the fluid comprising a predetermined pH.

### BRIEF DESCRIPTION OF THE DRAWINGS

This invention will be more fully understood from the following detailed description taken in conjunction with the accompanying drawings, in which:

FIG. 1 is a perspective view of one exemplary embodiment of a conventional surgical stapling and severing instrument.

FIG. 2A is a top view of a staple cartridge for use with the surgical stapling and severing instrument of FIG. 1;

FIG. 2B is a side view of the staple cartridge of FIG. 2A;

FIG. 3 is a side view of a staple in an unfired (pre-deployed) configuration that can be disposed within the staple cartridge of the surgical cartridge assembly of FIG. 2A;

FIG. 4 is a perspective view of a knife and firing bar (“E-beam”) of the surgical stapling and severing instrument of FIG. 1;

FIG. 5 is a perspective view of a wedge sled of a staple cartridge of the surgical stapling and severing instrument of FIG. 1;

FIG. 6A is a longitudinal cross-sectional view of an exemplary surgical cartridge assembly having a compressible non-fibrous adjunct attached to a top or deck surface of a staple cartridge;

FIG. 6B is a longitudinal cross-sectional view of a surgical end effector having an anvil pivotably coupled to an elongate channel and the surgical cartridge assembly of FIG. 6A disposed within and coupled to the elongate channel, showing the anvil in a closed position without any tissue between the anvil and the adjunct;

FIG. 7A is a partial-schematic illustrating the adjunct of FIGS. 6A-6B in a tissue deployed condition;

FIG. 7B is a diagram showing an enlarged portion of an exemplary adjunct with a porous structure;

FIG. 8 is a perspective view of an exemplary cartridge assembly;

FIG. 9A is a side view of an exemplary end effector having an adjunct of bioabsorbable material in a delivery configuration;

FIG. 9B is a side view of an exemplary end effector having an adjunct of bioabsorbable material after firing and release from cartridge;

FIG. 10A is a top perspective view of an exemplary adjunct after use;

FIG. 10B is a side view of an exemplary adjunct after use; and

FIG. 11 is a flow chart showing an exemplary method of forming a surgical adjunct with tunable absorption pathways in bioabsorbable material.

FIG. 12 is a flow chart showing an exemplary method of forming a surgical adjunct with tunable absorption pathways in bioabsorbable material.

### DETAILED DESCRIPTION

The following detailed description should be read with reference to the drawings, in which like elements in different drawings are identically numbered. The drawings, which are not necessarily to scale, depict selected embodiments and are not intended to limit the scope of the invention. The detailed description illustrates by way of example, not by way of limitation, the principles of the invention. This description will clearly enable one skilled in the art to make and use the invention, and describes several embodiments, adaptations, variations, alternatives and uses of the invention, including what is presently believed to be the best mode of carrying out the invention.

As used herein, the terms “about” or “approximately” for any numerical values or ranges indicate a suitable dimensional tolerance that allows the part or collection of components to function for its intended purpose as described herein. More specifically, “about” or “approximately” may refer to the range of values±10% of the recited value, e.g., “about 90%” may refer to the range of values from 81% to 99%.

As used herein, “degradation” refers to a process of breaking down of a polymer chain for absorption as atoms, molecules, or ions into the surroundings (e.g., tissue, cells, fluid, and the like). Degradation includes breaking primary and secondary bonds via thermal degradation, photodegradation, catalytic or enzymatic degradation, oxidative degradation, ion degradation, and biodegradation.

The term “polymerizable compound” means a compound containing one or more polymerizable groups. The term encompasses, for instance, monomers, macromers, oligomers, prepolymers, cross-linkers, and the like.

As used herein, “polymerizable groups” are groups that can undergo chain growth polymerization, such as free radical and/or cationic polymerization, for example a carbon-carbon double bond which can polymerize when subjected to radical polymerization initiation conditions. Non-limiting examples of free radical polymerizable groups include (meth)acrylates, styrenes, vinyl ethers, (meth)acrylamides, N-vinyllactams, N-vinylamides, O-vinylcarbamates, O-vinylcarbonates, and other vinyl groups. Preferably, the free radical polymerizable groups comprise (meth)acrylate, (meth)acrylamide, N-vinyl lactam, N-vinylamide, polyester polyols, poloxamers, and styryl functional groups, and mixtures of any of the foregoing. Preferably, the free radical polymerizable groups include (meth)acrylates, (meth)acrylamides, polyester polyols, poloxamers, and mixtures thereof. The polymerizable group may be unsubstituted or substituted. For instance, the nitrogen atom in (meth)acrylamide may be bonded to a hydrogen, or the hydrogen may be replaced with alkyl or cycloalkyl (which themselves may be further substituted).

The term “polyurethane,” as used herein, refers to a polymeric reaction product of an isocyanate and a polyol, and is not limited to those polymers which include only urethane or polyurethane linkages. It is well understood by those of ordinary skill in the art of preparing polyurethanes that the polyurethane polymers may also include linkages such as allophanate, carbodiimide, and other linkages described herein in addition to urethane linkages.

Any type of free radical polymerization may be used including but not limited to bulk, solution, suspension, and emulsion as well as any of the controlled radical polymerization methods such as stable free radical polymerization, nitroxide-mediated living polymerization, atom transfer radical polymerization, reversible addition fragmentation chain transfer polymerization, organotellurium mediated living radical polymerization, and the like.

The expressions “reaction system,” “reactive formulation,” “reaction product,” and “reactive mixture” are interchangeably used herein, and all refer to a combination of reactive compounds used to make the bioabsorbable material according to the disclosure.

The term “room temperature” refers to temperatures of about 20° C., this means referring to temperatures in the range 18° C. to 25° C. Such temperatures will include 18° C., 19° C., 20° C., 21° C., 22° C., 23° C., 24° C. and 25° C.

Unless otherwise expressed, the “weight percentage” (indicated as % wt. or wt. %) of a component in a composition refers to the weight of the component over the total weight of the composition in which it is present and is expressed as percentage.

“Glass transition temperature” and “T_{g}” as referred to herein refers to the temperature at which a reversible transition from a hard glass condition into a rubber-elastic condition occurs.

Surgical stapling assemblies and methods for manufacturing and using the same are provided. In general, a surgical stapling assembly can include a staple cartridge having staples disposed therein and an adjunct configured to be releasably retained on the staple cartridge. As discussed herein, the various adjuncts provided can be configured to compensate for variations in tissue properties, such as variations in tissue thickness, and/or to promote tissue ingrowth when the adjuncts are stapled to tissue. As discussed herein, an adjunct can include a bioabsorbable material, such as a foam.

An exemplary stapling assembly can include a variety of features to facilitate application of a surgical staple, as described herein and illustrated in the drawings. However, a person skilled in the art will appreciate that the stapling assembly can include only some of these features and/or it can include a variety of other features known in the art. The stapling assemblies described herein are merely intended to represent certain exemplary examples. Moreover, while the adjuncts are described in connection with surgical staple cartridge assemblies, the adjuncts can be used in connection with staple reloads that are not cartridge based or any type of surgical instrument.

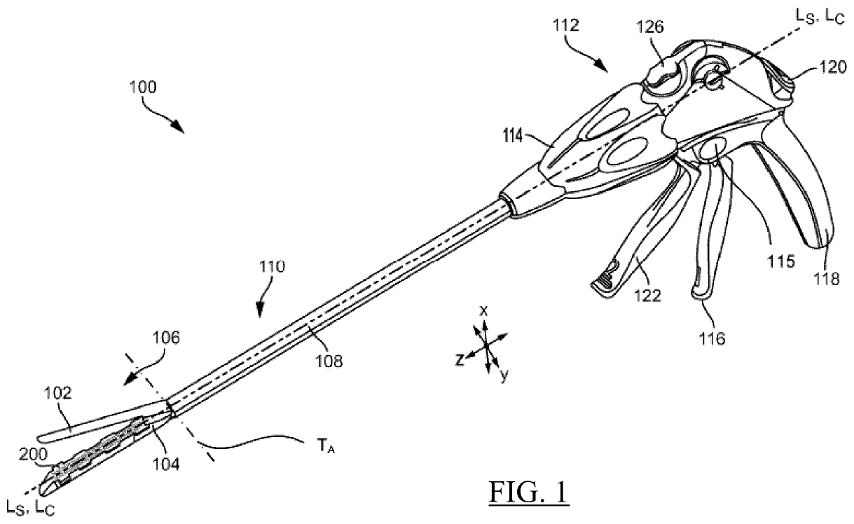

FIG. 1 illustrates an exemplary surgical stapling and severing device 100 suitable for use with an implantable adjunct. The illustrated surgical stapling and severing device 100 includes end effector 106 having an anvil 102 that is pivotably coupled to an elongate channel 104. As a result, the end effector 106 can move between an open position, as shown in FIG. 1, and a closed position in which the anvil 102 is positioned adjacent to the elongate channel 104 to engage tissue therebetween. The end effector 106 can be attached at its proximal end to an elongate shaft 108 forming an implement portion 110. When the end effector 106 is closed, or at least substantially closed, (e.g., the anvil 102 moves from the open position in FIG. 1 toward the elongate channel) the implement portion 110 can present a sufficiently small cross-section suitable for inserting the end effector 106 through a trocar. While the device 100 is configured to staple and sever tissue, surgical devices configured to staple but not sever tissue are also contemplated herein.

In various instances, the end effector 106 can be manipulated by a handle 112 connected to the elongate shaft 108. The handle 112 can include user controls such as a rotation knob 114 that rotates the elongate shaft 108 and the end effector 106 about a longitudinal axis (Ls) of the elongate shaft 108 and an articulation control 115 that can articulate the end effector 106 about an articulate axis (TA) that is substantially transverse to the longitudinal axis (Ls) of the elongate shaft 108. Further controls include a closure trigger 116 which can pivot relative to a pistol grip 118 to close the end effector 106. A closure release button 120 can be outwardly presented on the handle 112 when the closure trigger 116 is clamped such that the closure release button 120 can be depressed to unclamp the closure trigger 116 and open the end effector 106, for example. Handle 112 may also take the form of an interface for connection to a surgical robot.

In some examples, a firing trigger 122, which can pivot relative to the closure trigger 116, can cause the end effector 106 to simultaneously sever and staple tissue clamped therein. The firing trigger 122 may be powered, require force from a user to engage, or some combination thereof. A manual firing release lever 126 can allow the firing system to be retracted before full firing travel has been completed, if desired, and, in addition, the firing release lever 126 can allow a surgeon, or other clinician, to retract the firing system in the event that the firing system binds and/or fails.

Additional details on the surgical stapling and severing device 100 and other surgical stapling and severing devices suitable for use with the present disclosure are described, for example, in U.S. Pat. No. 9,332,984 and in U.S. Patent Publication No. 2009/0090763, the disclosures of which are incorporated herein by reference in their entireties. Further, the surgical stapling and severing device need not include a handle, but instead can have a housing that is configured to couple to a surgical robot, for example, as described in U.S. Patent Publication No. 2019/0059889, the disclosure of which is incorporated herein by reference in its entirety.

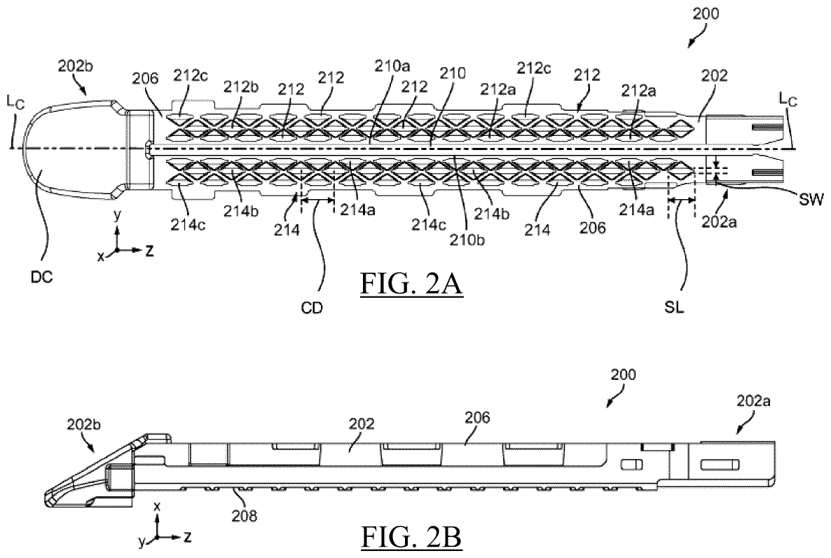

As further shown in FIG. 1, a staple cartridge 200 can be utilized with the instrument 100. In use, the staple cartridge 200 is placed within and coupled to the elongate channel 104. While the staple cartridge 200 can have a variety of configurations, in this illustrated example, the staple cartridge 200, which is shown in more detail in FIGS. 2A-2B, has a proximal end 202a and a distal end 202b with a cartridge longitudinal axis (LC) extending therebetween. As a result, when the staple cartridge 200 is inserted into the elongate channel 104 (FIG. 1), the longitudinal axis (LC) is substantially or approximately parallel with the longitudinal axis (LS) of the elongate shaft 108. Further, the staple cartridge 200 includes a longitudinal slot 210 defined by two opposing walls 210a, 210b and configured to receive at least a portion of a firing member of a firing assembly, like firing assembly 400 in FIG. 4, as discussed further below. As shown, the longitudinal slot 210 extends from the proximal end 202a toward the distal end 202b of the staple cartridge 200. It is also contemplated herein that in other examples, the longitudinal slot 210 can be omitted.

The illustrated staple cartridge 200 includes staple cavities 212, 214 defined therein, in which each staple cavity 212, 214 is configured to removably house at least a portion of a staple (not shown). The number, shape, and position of the staple cavities can vary and can depend at least on the size and shape (e.g., mouth-like shape) of the staples to be removably disposed therein. In this illustrated example, the staple cavities are arranged in two sets of three longitudinal rows, in which the first set of staple cavities 212 is positioned on a first side of the longitudinal slot 210 and the second set of staple cavities 214 is positioned on a second side of the longitudinal slot 210. On each side of the longitudinal slot 210, and thus for each set of rows, a first longitudinal row of staple cavities 212a, 214a extends alongside the longitudinal slot 210, a second row of staple cavities 212b, 214b extends alongside the first row of staple cavities 212a, 214a, and a third row of staple cavities 212c, 214c extends alongside the second row of staple cavities 212b, 214b. Each row may be approximately parallel and the staple cavities that make up the rows may be approximately parallel in orientation with the longitudinal slot 210. As shown in FIGS. 2A, each staple cavity 212, 214 may include a maximum length SL of about 0.122 inches to about 0.124 inches and a maximum width SW of about 0.023 inches to about 0.027 inches. In addition, at least the centers of two adjacent cavities 212, 214 are spaced apart by about 0.158 inches.

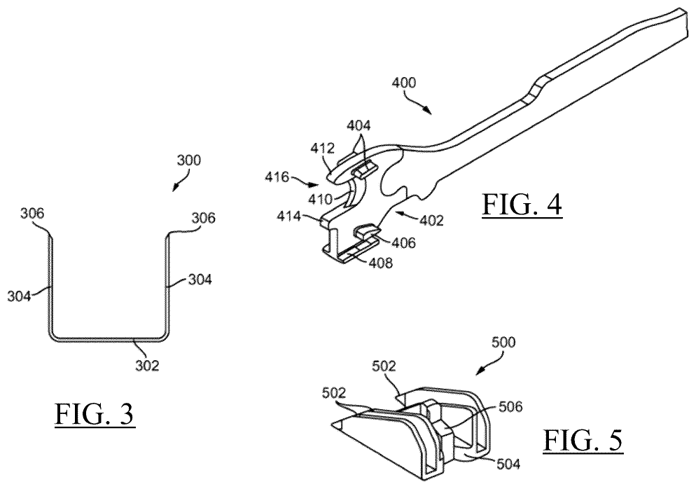

The staples releasably stored in the staple cavities 212, 214 can have a variety of configurations. An exemplary staple 300 that can be releasably stored in each of the staple cavities 212, 214 is illustrated in FIG. 3 in its unfired (pre-deployed, unformed) configuration. The illustrated staple 300 includes a crown (base) 302 and two legs 304 extending from each end of the crown 302. In this example, the crown 302 extends in a linear direction and the staple legs 304 have the same unformed height. Further, prior to the staples 300 being deployed, the staple crowns 302 can be supported by staple drivers that are positioned within the staple cartridge 200 and, concurrently, the staple legs 304 can be at least partially contained within the staple cavities 212, 214. Further, the staple legs 304 can extend beyond a top surface, like top surface 206, of the staple cartridge 200 when the staples 300 are in their unfired positions. In certain instances, as shown in FIG. 3, the tips 306 of the staple legs 304 can be pointed and sharp which can incise and penetrate tissue.

In use, staples 300 can be deformed from an unfired position into a fired position such that the staple legs 304 move through the staple cavities 212, 214, penetrate tissue positioned between the anvil 102 and the staple cartridge 200, and contact the anvil 102. As the staple legs 304 are deformed against the anvil 102, the legs 304 of each staple 300 can capture a portion of the tissue within each staple 300 and apply a compressive force to the tissue. Further, the legs 304 of each staple 300 can be deformed downwardly toward the crown 302 of the staple 300 to form a staple entrapment area in which the tissue can be captured therein. In various instances, the staple entrapment area can be defined between the inner surfaces of the deformed legs and the inner surface of the crown of the staple. The size of the entrapment area for a staple can depend on several factors such as the length of the legs, the diameter of the legs, the width of the crown, and/or the extent in which the legs are deformed, for example.

In some examples, all of the staples disposed within the staple cartridge 200 can have the same unfired (pre-deployed, unformed) configuration. In other examples, the staples can include at least two groups of staples each having a different unfired (pre-deployed, unformed) configuration, e.g., varying in height and/or shape, relative to one another, etc.

Referring back to FIGS. 2A-2B, the staple cartridge 200 extends from a top surface or deck surface 206 to a bottom surface 208, in which the top surface 206 is configured as a tissue-facing surface and the bottom surface 208 is configured as a channel-facing surface. As a result, when the staple cartridge 200 is inserted into the elongate channel 104, as shown in FIG. 1, the top surface 206 faces the anvil 102 and the bottom surface 208 (obstructed) faces the elongate channel 104.

With reference to FIGS. 4 and 5, a firing assembly such as, for example, firing assembly 400, can be utilized with a surgical stapling and severing device, like device 100 in FIG. 1. The firing assembly 400 can be configured to advance a wedge sled 500 having wedges 502 configured to deploy staples from the staple cartridge 200 into tissue captured between an anvil, like anvil 102 in FIG. 1, and a staple cartridge, like staple cartridge 200 in FIG. 1. Furthermore, an E-beam 402 at a distal portion of the firing assembly 400 may fire the staples from the staple cartridge. During firing, the E-beam 402 can also cause the anvil to pivot towards the staple cartridge, and thus move the end effector from the open position towards a closed position. The illustrated E-beam 402 includes a pair of top pins 404, a pair of middle pins 406, which may follow a portion 504 of the wedge sled 500, and a bottom pin or foot 408. The E-beam 402 can also include a sharp cutting edge 410 configured to sever the captured tissue as the firing assembly 400 is advanced distally, and thus towards the distal end of the staple cartridge. In addition, integrally formed and proximally projecting top guide 412 and middle guide 414 bracketing each vertical end of the cutting edge 410 may further define a tissue staging area 416 assisting in guiding tissue to the sharp cutting edge 410 prior to being severed. The middle guide 414 may also serve to engage and fire the staples within the staple cartridge by abutting a stepped central member 506 of the wedge sled 500 that effects staple formation by the end effector 106.

In use, the anvil 102 in FIG. 1 can be moved into a closed position by depressing the closure trigger in FIG. 1 to advance the E-beam 402 in FIG. 4. The anvil 102 can position tissue against at least the top surface 206 of the staple cartridge 200 in FIGS. 2A-2B. Once the anvil has been suitably positioned, the staples 300 in FIG. 3 disposed within the staple cartridge can be deployed.

To deploy staples from the staple cartridge, as discussed above, the sled 500 in FIG. 5 can be moved from the proximal end toward a distal end of the cartridge body, and thus, of the staple cartridge. As the firing assembly 400 in FIG. 4 is advanced, the sled can contact and lift staple drivers within the staple cartridge upwardly within the staple cavities 212, 214. In at least one example, the sled and the staple drivers can each include one or more ramps, or inclined surfaces, which can co-operate to move the staple drivers upwardly from their unfired positions. As the staple drivers are lifted upwardly within their respective staple cavities, the staples are advanced upwardly such that the staples emerge from their staple cavities and penetrate into tissue. In various instances, the sled can move several staples upwardly at the same time as part of a firing sequence.

As indicated above, the stapling device can be used in combination with a compressible adjunct. A person skilled in the art will appreciate that, while adjuncts are shown and described below, the adjuncts disclosed herein can be used with other surgical instruments and need not be coupled to a staple cartridge as described. Further, a person skilled in the art will also appreciate that the staple cartridges need not be replaceable.

As discussed above, with some surgical staplers, a surgeon is often required to select the appropriate staples having the appropriate staple height for tissue to be stapled. For example, a surgeon will utilize tall staples for use with thick tissue and short staples for use with thin tissue. In some instances, however, the tissue being stapled does not have a consistent thickness and thus, the staples cannot achieve the desired fired configuration for every section of the stapled tissue (e.g., thick and thin tissue sections). The inconsistent thickness of tissue can lead to undesirable leakage and/or tearing of tissue at the staple site when staples with the same or substantially greater height are used, particularly when the staple site is exposed to intra-pressures at the staple site and/or along the staple line.

Accordingly, various examples of adjuncts are provided that can be configured to compensate for varying thickness of tissue that is captured within fired (deployed) staples to avoid the need to take into account staple height when stapling tissue during surgery. That is, the adjuncts described herein can allow a set of staples with the same or similar heights to be used in stapling tissue of varying thickness (e.g., from thin to thick tissue) while also, in combination with the adjunct, providing adequate tissue compression within and between fired staples. Thus, the adjuncts described herein can maintain suitable compression against thin or thick tissue stapled thereto to thereby minimize leakage and/or tearing of tissue at the staple sites. In addition, exemplary adjuncts described herein may be configured to be absorbed in the body over a period of 100 to 300 days depending on implanted location and tissue health.

Alternatively, or in addition, the adjuncts can be configured to promote tissue ingrowth. In various instances, it is desirable to promote the ingrowth of tissue into an implantable adjunct, to promote the healing of the treated tissue (e.g., stapled and/or incised tissue), and/or to accelerate the patient's recovery. More specifically, the ingrowth of tissue into an implantable adjunct may reduce the incidence, extent, and/or duration of inflammation at the surgical site. Tissue ingrowth into and/or around the implantable adjunct may, for example, manage the spread of infections at the surgical site. The ingrowth of blood vessels, especially white blood cells, for example, into and/or around the implantable adjunct may fight infections in and/or around the implantable adjunct and the adjacent tissue. Tissue ingrowth may also encourage the acceptance of foreign matter (e.g., the implantable adjunct and the staples) by the patient's body and may reduce the likelihood of the patient's body rejecting the foreign matter. Rejection of foreign matter may cause infection and/or inflammation at the surgical site.

In general, the adjuncts provided herein are designed and positioned atop a staple cartridge, like staple cartridge 200. When the staples are fired (deployed) from the cartridge, the staples penetrate through the adjunct and into tissue. As the legs of the staple are deformed against the anvil that is positioned opposite the staple cartridge, the deformed legs capture a portion of the adjunct and a portion of the tissue within each staple. That is, when the staples are fired into tissue, at least a portion of the adjunct becomes positioned between the tissue and the fired staple. While the adjuncts described herein can be configured to be attached to a staple cartridge, it is also contemplated herein that the adjuncts can be configured to mate with other instrument components, such as an anvil of a surgical stapler. A person of ordinary skill will appreciate that the adjuncts provided herein can be used with replaceable cartridges or staple reloads that are not cartridge based.

In various embodiments, the adjunct or bioabsorbable materials disclosed herein can be comprised of an absorbable polymer. In certain embodiments, an adjunct can be comprised of foam, film, fibrous woven, fibrous non-woven polyurethane, polyether urethane, polyester urethane, polyester urea, polyester, polycarbonate, polyorthoester, polyanhydride, polyesteramide, polyphosphazenes, polyphosphoesters, polysaccharides, and/or polyoxaester. In other embodiments, an adjunct can be a copolymer including, for example, PGA (polyglycolic acid), PGA/PCL (poly(glycolic acid-co-caprolactone)), PLA/PCL (poly(lactic acid-co-polycaprolactone)), PLLA/PCL, PGA/TMC (poly(glycolic acid-co-trimethylene carbonate)), PDS, PEPBO, and the like. In various embodiments, an adjunct can include an organic material such as, for example, carboxymethyl cellulose, sodium alginate, hyaluronic acid, and/or oxidized regenerated cellulose. In various embodiments, an adjunct has a durometer in the 3-7 Shore A (30-50 Shore OO) ranges with a maximum stiffness of 15 Shore A (65 Shore OO). In certain embodiments, an adjunct can undergo 40% compression under 3 lbf load, 60% compression under 6 lbf load, and/or 80% compression under 20 lbf load, for example. In certain embodiments, one or more gasses, such as air, nitrogen, carbon dioxide, and/or oxygen, for example, can be bubbled through and/or contained within the adjunct.

### Methods of Stapling Tissue

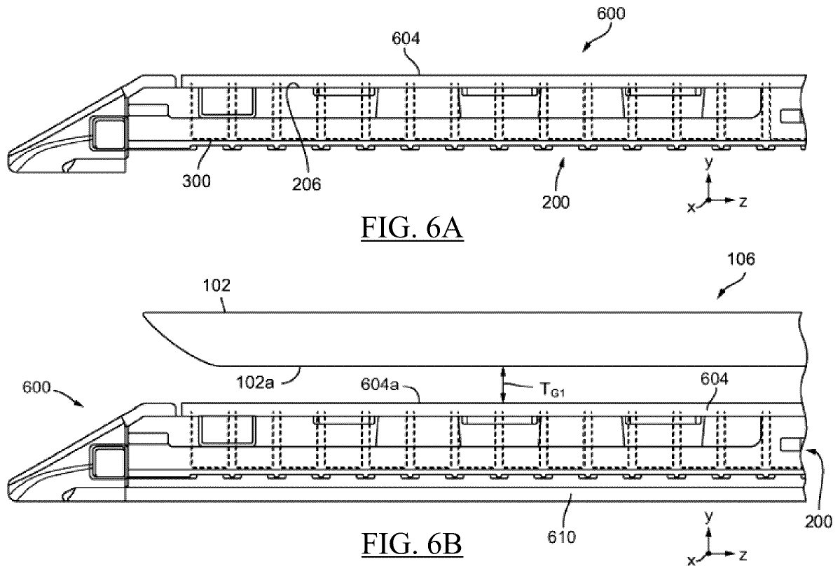

FIGS. 6A-6B illustrate an exemplary example of a stapling assembly 600 that includes a staple cartridge 200 and an adjunct 604. For sake of simplicity, the adjunct 604 is generally illustrated in FIGS. 6A-6B, and various configurations of the adjunct are described in more detail below. As shown, the adjunct 604 is positioned against the staple cartridge 200. While partially obstructed in FIGS. 6A-6B, the staple cartridge 200 includes staples 300, that are configured to be deployed into tissue. The staples 300 can have any suitable unformed (pre-deployed) height.

In the illustrated example, the adjunct 604 can be mated to at least a portion of the top surface or deck surface 206 of the staple cartridge 602. In some examples, the top surface 206 of the staple cartridge 200 can include one or more surface features which can be configured to engage the adjunct 604 to avoid undesirable movements of the adjunct 604 relative to the staple cartridge 200 and/or to prevent premature release of the adjunct 604 from the staple cartridge 200. Exemplary surface features are described further below and in U.S. Pat. No. 10,052,104, which is incorporated by reference herein in its entirety.

FIG. 6B shows the stapling assembly 600 placed within and coupled to the elongate channel 610 of surgical end effector 106. The anvil 102 is pivotally coupled to the elongate channel 610 and is thus moveable between open and closed positions relative to the elongate channel 610, and thus the staple cartridge 200. The anvil 102 is shown in a closed position in FIG. 6B and illustrates a tissue gap T_{G1} created between the staple cartridge 602 and the anvil 612. More specifically, the tissue gap T_{G1} is defined by the distance between the tissue-compression surface 102a of the anvil 102 (e.g., the tissue-engaging surface between staple forming pockets in the anvil) and the tissue-contacting surface 604a of the adjunct 604. In this illustrated example, both the tissue-compression surface 102a of the anvil 102 and the tissue-contacting surface 604a of the adjunct 604 is planar, or substantially planar (e.g., planar within manufacturing tolerances). As a result, when the anvil 102 is in a closed position, as shown in FIG. 6B, the tissue gap T_{G1} is generally uniform (e.g., nominally identical within manufacturing tolerances) when no tissue is disposed therein. In other words, the tissue gap T_{G1} is generally constant (e.g., constant within manufacturing tolerances) across the end effector 106 (e.g., in the y-direction). In other examples, the tissue-compression surface of the anvil can include a stepped surface having longitudinal steps between adjacent longitudinal portions, and thus create a stepped profile (e.g., in the y-direction). In such examples, the tissue gap T_{G1} can be varied.

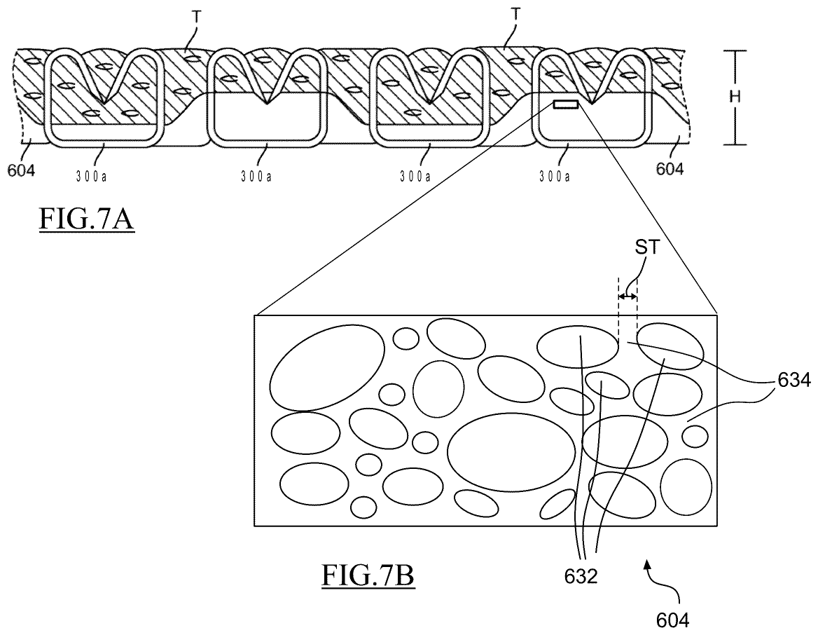

The adjunct 604 is compressible to permit the adjunct to compress to varying heights to thereby compensate for different tissue thickness that are captured within a deployed staple. The adjunct 604 has an uncompressed (undeformed), or pre-deployed, height and is configured to deform to one of a plurality of compressed (deformed), or deployed, heights. For example, the adjunct 604 can have an uncompressed height which is greater than the fired height of the staples 300 disposed within the staple cartridge 200 (e.g., the height (H) of the fired staple 300a in FIG. 7A). That is, the adjunct 604 can have an undeformed state in which a maximum height of the adjunct 604 is greater than a maximum height of a fired staple (e.g., a staple that is in a formed configuration).

In use, once the surgical stapling and severing device, like device 100 in FIG. 1, is directed to the surgical site, tissue is positioned between the anvil 102 and the stapling assembly 600 such that the anvil 102 is positioned adjacent to a first side of the tissue and the stapling assembly 600 is positioned adjacent to a second side of the tissue (e.g., the tissue can be positioned against the tissue-contacting surface 604a of the adjunct 604). Once tissue is positioned between the anvil 102 and the stapling assembly 600, the surgical stapler can be actuated, e.g., as discussed above, to thereby clamp the tissue between the anvil 102 and the stapling assembly 600 (e.g., between the tissue-compression surface 102a of the anvil 102 and the tissue-contacting surface 604a of the adjunct 604) and to deploy staples from the cartridge through the adjunct and into the tissue to staple and attach the adjunct to the tissue.

As shown in FIG. 7A, when the staples 300 are fired, tissue (T) and a portion of the adjunct 604 are captured by the fired (formed) staples 300a. The fired staples 300a each define the entrapment area therein, as discussed above, for accommodating the captured adjunct 604 and tissue (T). The entrapment area defined by a fired staple 300a is limited, at least in part, by a height (H) of the fired staple 300a.

Referring to FIG. 7B, the adjunct 604 may have pores 632 with a median pore size of about 0.025 mm³ to about 0.300 mm³, such as about 0.022 mm³. In some examples, the adjunct 604 may have one or more struts 634 between the pore 632 that provide support and strength to the adjunct 604. In particular, the adjunct 604 may include a plurality of struts 634, having a median strut thickness ST of about 0.025 mm to about 0.300 mm, such as about 0.08 mm.

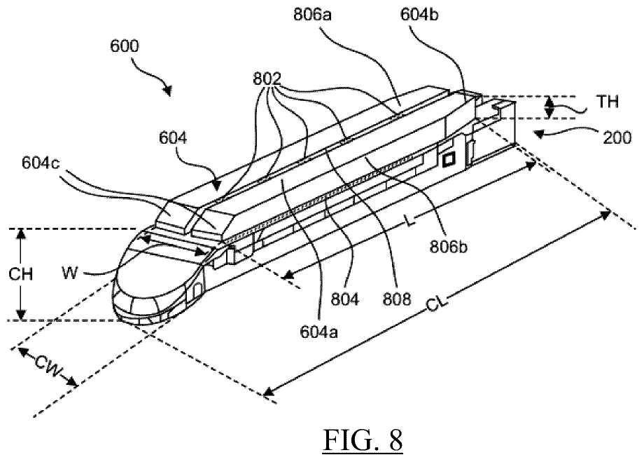

FIG. 8 illustrates a perspective view of a staple cartridge assembly 600 with an adjunct 604 and a staple cartridge 200. The adjunct 604 has a tissue contacting surface 604a, a proximal end 604c, and a distal end 604b. The adjunct 604 may include a slot/slit 808 separating or partially separating two parallel portions of the adjunct 604. In one example, adjunct 604 may include a slot 808 separating two parallel portions of the adjunct 604, while in another example, adjunct 604 may include a slit 808 separating two parallel portions of the adjunct 604 and also one or more bridges (e.g., five bridges) 802 connecting the two parallel portions of the adjunct 604. At least one bridge has a length in the longitudinal direction of about 0.035 inches to about 0.046 inches. The adjunct 604 has a length L of about 40 mm to about 80 mm, such as about 60 mm to about 65 mm, about 66.04 mm to about 66.3 mm, about 45 mm to about 55 mm, or about 51.12 mm to about 51.38 mm. The adjunct 604 has a width W of about 8 mm to about 12 mm, such as about 9.75 mm to about 10.25 mm or about 10.025 mm to about 10.035 mm. The adjunct 604 may also have a thickness or height TH of about 2.5 mm to about 3.5 mm, such as about 2.85 mm to about 3.15 mm or about 2.95 mm to about 3.05 mm.

The cartridge 200 has a height CH of about 6.3 mm to about 8.1 mm, a width CW of about 8.9 mm to about 14 mm, and a length CL of about 80 mm to about 90 mm such as about 86.7 mm.

The staple cartridge 200 may include one or more raised ledges 804 along one or more sides of the adjunct 604 to help align the adjunct 604 on the deck of the staple cartridge 200. Although not shown in FIG. 8, the staple cartridge 200 may also include an adhesive or buttress adhesive material to attach the adjunct 604. The adjunct 604 may be attached to the cartridge 200 with about 100 mg to about 120 mg of the adhesive or buttress adhesive material.

### Tunable Absorption Pathways for Bioabsorbable Material

Balancing mechanical property depletion with backbone absorption of the adjunct after delivery may be improved with a dual phase degradation mechanism. As described above, the end effector 106 (shown in FIG. 1) comprising the cartridge 200 and adjunct 604 is closed or substantially closed for inserting through a trocar to the delivery site. Therefore, the surgical adjunct 604 needs to have mechanical properties such that the material is sufficiently compressible during delivery to be inserted through a trocar, but must have increased strength to retain staples, sutures, and screws at the delivery site. In addition, the surgical adjunct 604 needs to be bioabsorbable such that after delivery, the adjunct 604 remains at the target site until the tissue can sufficiently maintain a hemostatic seal and/or the tissue is sufficiently healed. From a mechanical standpoint, the adjunct 604 provides an ideal amount of compression that is both strong enough to create and maintain the hemostatic seal and is not over-compressing or too tough to permit firing of the staples upon delivery. A short-term absorption profile may be preferred to address hemostasis while a long-term absorption profile may address better tissue healing without leakages.

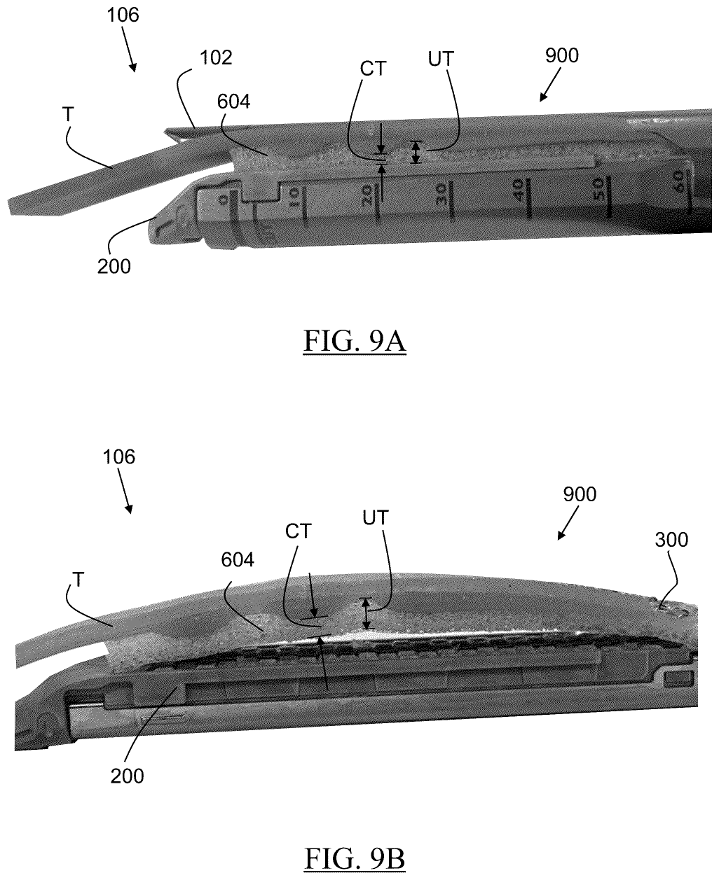

Turning to FIGS. 9A and 9B, an example staple cartridge assembly 900 includes an end effector 106 having a surgical adjunct 604 and a staple cartridge 200. The surgical adjunct 604 can be composed of a substantially monolithic structure. In addition, the adjunct 604 can be a bioabsorbable material that includes a first polymerizable compound that has a first degradation profile. The material choice of the first polymerizable compound can be a reaction product of a polyol and an isocyanate, such as polyurethane or a like polymer including, without limitation, polyether urethane, polyester urethane, polyester urea, polyester, polycarbonate, polyorthoester, polyanhydride, polyesteramide, polyphosphazenes, polyphosphoesters, polysaccharides, and/or polyoxaester. The polyurethane reaction product can have a degradation profile that is tailored for the target tissue site. The surgical adjunct 604 can be tuned the particular purpose before, during, and after surgical procedures and have one or more of the properties described below that modulate the mechanical properties. In particular, the adjunct 604 described has selective crosslinking density control in vivo and can also have a certain compressibility when attached to the cartridge outside of the body.

In some embodiments, the first polymerizable compound can be a reaction product of a polyol and an isocyanate, forming a polyurethane or polyurethane derivate. The polyurethane is configured to degrade according to a first degradation profile. An aliphatic polyurethane primarily undergoes oxidative and/or enzymatic mechanisms of absorption. To maintain the mechanical properties when firing the staples while tuning the absorption profile after minutes, hours, days, or weeks after delivery, a second polymerizable compound is added. The second polymerizable compound is configured to degrade according to a second degradation profile. In some examples, the second polymerizable compound can adjust an induction period for absorption, thereby delaying bulk absorption of the adjunct 604. In some instances, the physiological conditions at the delivery site can be used to accelerate absorption of at least one of the first polymerizable compound and/or the second polymerizable compound.

As described herein, the surgical adjunct 604 can be tuned for the particular purpose before, during, and after surgical procedures. The adjunct 604 includes a first polymerizable compound that has a first degradation profile and a second polymerizable compound with a second degradation profile different than the first. In particular, the adjunct 604 described can be a polymer with tunable absorption properties via an alternating copolymer, a random copolymer, a block copolymer, a multiblock copolymer architecture, a terpolymer, a graft copolymer (monomer backbone with pendant chains of a different monomer), a bulk homopolymer coated with a second homopolymer, and the like.

In some embodiments, the second polymerizable compound can be a polyester, a poloxamer, a polymethacrylate, a polyether, a polydioxanone, a polyanhydride, a hydroxypropyl methylcellulose acetate succinate, a cellulose acetate phthalate, a cellulose acetate trimellitate, a hydroxypropyl methylcellulose phthalate, a polyvinyl acetate phthalate, a poly(trimethylene carbonate), a poly(beta-thioether ester ketal), a polypropylene fumarate, a poly(ester urea), a poly(ester amide).

In some embodiments, the first polymerizable compound alone has a suitable degradation profile for healing tissue within approximately 6 weeks. In some embodiments, it is desirable to tune the degradation profile of the adjunct 604 for longer degradation profiles. In some embodiments, the bioabsorption rate of the adjunct 604 can be tuned by adding more or less of the second polymerizable compound to the first polymerizable compound. In general, the adjunct 604 can have approximately equal parts of the first polymerizable compound to second polymerizable compound. In certain other embodiments, the volumetric ratio of first polymerizable compound to second polymerizable compound can range from about 10:0.1 to about 0.1:10. For instance, when it is desirable to have a longer degradation profile, the volumetric ratio can be approximately 10:1, about 10:2, about 10:3, or about 10:4. As another non-limiting example, to tune the degradation profile to shorter periods of time, the volumetric ratio can be approximately 1:10, approximately 2:10, approximately 3:10, or approximately 4:10. As would be appreciated by one of skill in the relevant art, tuning the degradation profile of the adjunct as a whole can be done through adjusting the type, molecular weight, and quantity of first polymerizable compound to second polymerizable compound.

In some embodiments, the second polymerizable compound can be added to the polyurethane backbone such that the first and second polymerizable compounds together form a copolymer backbone. When the second polymerizable compound is added to the polyurethane backbone, the second polymerizable compound can be one of the polyol or the isocyanate. For instance, the second polymerizable compound can be a polyester polyol that when added to the first polymerizable compound, the polyester polyol can replace the polyol of the polyurethane reaction product to form a block copolymer or an alternating copolymer. Alternatively, or in addition thereto, the polyester polyol can be in addition to the polyol of the polyurethane reaction product such that more than one type of polyol monomer is introduced into the backbone of the polyurethane, such as in a block copolymer (A-B-A style) or a random copolymer.

The inclusion of an aliphatic polyester polyol as the second polymerizable compound, can add another degree of freedom in a polyurethane backbone to tune the absorption and mechanical properties of the adjunct 604. Above a certain molecular weight, mechanical toughening can occur through crystallization that effectively adds crosslinks to the polyurethane system and thus allow for lower covalent crosslinks overall. In general, reducing the number of covalent crosslinks can result in an accelerated degradation profile of the adjunct 604.

In addition to the change in crosslinking density, the addition of the second polymerizable compound such as polyester polyols, can increase the backbone susceptibility to hydrolysis mechanism of absorption. Under a hydrolysis mechanism pathway, the sensitivity to acidic conditions could be used to preferentially increase the absorption rate or degradation profile of the adjunct 604 in the presence of lower pH.

The typical polyester polyol is branched with a weight average molecular weight (M_{w}) of 2,000-10,000. Polyester polyols are typically made from mixtures of diols, triols, and dibasic acids or anhydrides. Polyether polyols are made by the reaction of epoxides with compounds having active hydrogen atom. Polyester polyols are made by the polycondensation reaction of multifunctional carboxylic acids and polyhydroxyl compounds. Example polyester polyol monomers include, without limitation, poly(hexamethylene adipate) (PHA), aminocaproic acid, hexamethylenediamine adipate (1:1), 6-aminiohexanoate, 1,4-butanediamine adipate, hexanedioic acid undecane-1,11-diamine, 6-diaminohexanoic acid, calcium bis(6-aminohexanoate), 6-aminohexanoyloxidanium, 6-aminohexanoic acid, octane-1,8-diamine hexanedioic acid, ethane-1,1,2-triamine hexanedioic acid, pentane-1,5-diamine heptanedioic acid, hexane-1,1-diamine hexanedioic acid, butane-1,1-diamine hexanedioic acid, (2S)-2,6-diaminohexanoic acid hexanedioic acid, heptane-1,6-diamine hexanedioic acid, 2-aminoacetic acid 2,6-diaminohexanoic acid, 2,6-diaminohexanoic acid hexanedioic acid, 2-aminoacetic acid (2S)-2,6-diaminohexanoic acid, cadaverine adipate, cadaverine adipate dihydrate, hexanedioic acid nonane-1,9-diamine, 6-aminocaproic acid-d6, 6-amino-2,2,6,6-tetradeuteriohexanoic acid, 6-aminohexanoic acid butanedioic acid, 6-amino-6,6-dideuteriohexanoic acid, 6-amino-2,2-dideuteriohexanoic acid, hexanedioic acid propan-1-amine, hexane-1,6-diamine octanedioic acid, 6-aminohexanoic acid hexanedioic acid, 6-(6-aminohexanoyloxy)-6-oxohexanoic acid, hexan-1-amine hexanedioic acid, 6-aminohexanoic acid carbamic acid, 2-aminoacetic acid 6-aminohexanoic acid, 6-aminohexanoic acid 3-aminopropanoic acid, ethane-1,2-diamine hexanedioic acid, 7-aminoheptanoic acid hexane-1,6-diamine, 6-aminohexanoic acid (2s)-2,6-diaminohexanoic acid, 6-aminohexanoic acid 3-aminopropanoic acid, butane-1,4-diamine heptanedioic acid, 6-amino-6-oxohexanoic acid hexane-1,6-diamine, acetic acid 6-aminohexanoic acid, 6-aminohexanoic acid azane, ethanamine hexanedioic acid, 4-aminobutanoic acid 6-aminohexanoic acid, 6-aminohexanoyl 6-aminohexaneperoxoate, 6-aminohexanoic acid ethane-1,2-diamine, 5-azaniumylpentylazanium hexanedioate, heptane-1,1-diamine hexanedioic acid, amino hexanoate 6-aminohexanoic acid, decane-1,10-diamine hexanedioic acid, ethane-1,2-diamine hexanedioic acid, 6-azaniumylhexylazanium hexanedioate hexanedioic acid, 6-(dideuterioamino) hexanoic acid, 7-aminoheptanoic acid 6-aminohexanoic acid, hexane-1,5-diamine hexanedioic acid, 6-aminohexanoic acid hexanoic acid, hexamethylendiammonium-adipat-dihydrat, tetramethylene diammonium adipate, 6-aminohexanoic acid hexane-1,6-diamine, 6-(4-aminobutylamino)oxy-6-oxohexanoic acid, hexane-1,6-diamine 6-hydroxyhexanoic acid, 6-azaniumylhexylazanium hexanedioate hydrate, 6-aminohexanoic acid hydrate, 6-aminohexanoic acid methylideneazanium, 6-aminohexanoic acid ethane molecular hydrogen, 6-aminohexanoic acid methanamine, heptane-1,7-diamine heptanedioic acid, 6-aminohexanoic acid ethane fermium cyanide, 5-carboxypentylazanide, 6-(6-aminohexylamino)oxy-6-oxohexanoic acid, 6-aminohexanoic acid zinc, octanedioic acid pentane-1,5-diamine, azanium 6-aminohexanoic acid, 6-aminohexanoic acid 2,6-diaminohexanoic acid, carbamic acid hexane-1,6-diamine hexanedioic acid, 6-(6-aminohexylamino)oxy-6-oxohexanoic acid methane, azanium 6-azaniumylhexylazanium hexanedioate, butan-1-amine hexanedioic acid, heptan-1-amine hexanedioic acid, 6-aminohexanoic acid 8-aminooctanoic acid, 7-aminoheptanoic acid heptanedioic acid, hexanedioic acid pentane-1,1-diamine, N-(6-aminohexyl) hydroxylamine hexanedioic acid, 6-azaniumylhexylazanium hexanedioic acid, 6-aminohexanoic acid ethane, 6-aminohexanoic acid hydrate, 7-azaniumylheptylazanium hexanedioate, butane-1,4-diamine hexanoic acid, 4-aminobutanoic acid; 6-aminohexanoic acid; 3-aminopropanoic acid, and 6-aminohexanoate.

In some embodiments, an adjunct 604 that has a hydroxy-terminated polymer chain can enhance the biodegradation profile. A stoichiometric excess of hydroxyl over carboxylic acid functionality ensures that the finished polymer is hydroxy-terminated, whereas a carboxyl excess leads to carboxylic acid-terminated polyesters. Branching can be incorporated into the polyester backbone using trifunctional monomers such as trimethylolpropane, 2,2-di(hydroxymethyl)-1-butanol, or trimellitic anhydride.

Polyether polyols are the most common type of polyols. Polyether polyols are made by the reaction of epoxides with compounds having active hydrogen atom. The typical polyester polyol. Polyether polyols have low glass transition temperature (T_{g}), which imparts good retention of physical properties and impact resistance at very low temperatures. In general polyether-based polyurethanes exhibit higher rebound (resilience) compared to polyester-based polyurethanes. Example polyether polyol monomers include, without limitation, polyethylene glycol, polytetramethylene ether glycol, polypropylene oxide glycol, polybutylene oxide glycol, triethylene glycol monoethyl ether, 1,2-diethoxyethane 2-[2-(2-methoxyethoxy)ethoxy]ethanol, 1,2-diethoxyethane 2-methoxyethanol, 2-[2-[2-(2-hydroxyethoxy)ethoxy]ethoxy]ethanol methane, 1-ethoxy-2-methoxyethane 2-methoxyethanol, methane 2-[2-(2-methoxyethoxy)ethoxy]ethanol hydrate, methanol 2-[2-(2-methoxyethoxy)ethoxy]ethanol, 2-(2-(2-(2-(2-(2-ethoxyethoxy)ethoxy)ethoxy)ethoxy)ethoxy)ethanol, 2-[2-(2-ethoxyethoxy)ethoxy]ethanol hydrate, 2,5,8,11-tetraoxatridecan-13-ol, 2-(2-{2-[2-(2-methoxy-ethoxy)-ethoxy]-ethoxy}-ethoxy)-ethanol, 2,5,8,11,14,17-hexaoxanonadecan-19-ol, 3,6,9,12-tetraoxatetradecan-1-ol, 3,6,9,12,15-pentaoxaheptadecan-1-ol, 4,7,10,13-tetraoxa-1-oxoniacyclopentadecane, pentaethylene glycol-water, 2-[2-[2-[2-(2-ethoxyethoxy)ethoxy]ethoxy]ethoxy]ethanol, butane ethane-1,2-diol 2-propoxyethanol, butane 2-ethoxyethanol, ethanol, 2,2′-[1,2-ethanediylbis(oxy)]bis-, mixt. with 2,2′-oxybis [ethanol], 3,6,9,12,15-pentaoxaoctadecan-1-ol, 3,6,9,12-tetraoxapentadecan-1-ol, 2-(2-(2-propoxyethoxy)ethoxy) ethanol, pentaethylene glycol, hexaethylene glycol, tetraethylene glycol, triethylene glycol monomethyl ether, sodium 1,4,7,10,13,16-hexaoxoniacyclooctadecane, sodium 1,4,7,10,13-pentaoxoniacyclopentadecane, sodium 1,4,7,10-tetraoxoniacyclododecane, potassium 1,4,7,10,13-pentaoxoniacyclopentadecane, ytterbium 2-methoxyethoxide, cerium 2-methoxyethoxide, 2-[2-[2-(2-propoxyethoxy)ethoxy]ethoxy]ethanol, neodymium methoxyethoxide, erbium methoxyethoxide, ethanol 2-(2-hydroxyethoxy) ethanol pentane, butyl carbitol water, 2-[2-[2-(2-hydroxyethoxy)ethoxy]ethoxy]ethyl hypofluorite, lanthanum methoxyethoxide, 2-[2-(2-butoxyethoxy)ethoxy]ethanol propane, potassium 1,4,7,10,13,16-hexaoxoniacyclooctadecane, 2-[2-[2-[2-(2-hydroxyethynoxy)ethoxy]ethoxy]ethoxy]ethanol, 3,6,9,12,15,18-hexaoxahenicosan-1-ol, triethylene glycol-water, yttrium methoxyethoxide, heptaethylene glycol monomethyl ether, 2-(2-(2-ethoxyethoxy)ethoxy), 3,6,9,12,15,18-hexaoxaicosane-1,20-diol, 3,6,9,12,15-pentaoxanonadecan-1-ol, 3,6,9,12-tetraoxahexadecan-1-ol, triethylene glycol monobutyl ether, and the like.

Adding portions of the adjunct 604 with varying degrees of hydrophilicity can allow the adjunct to undergo a phase change upon exposure to a fluid. As the hydrophilicity increases, the adjunct will move from surface to bulk degradation due to the increased water ingress. In some examples, as the fluid swells the network, a material having the same polarity as the polymer may pass through a lower critical solution temperature (LCST). In general, the LCST is the critical temperature below which the components of a mixture are miscible in all proportions. In some embodiments, the polymer system in solution has an LCST at a temperature higher than the upper critical solution temperature (UCST), meaning that there is a temperature interval of complete miscibility, with partial miscibility at both higher and lower temperatures. The UCST is the critical temperature above which the components of a mixture are miscible in all proportions.

For polymers in a solution or fluid, the LCST depends on the polymer's degree of polymerization, polydispersity, branching, composition, and architecture. At temperatures below the LCST, portions of the polymer in solution or fluid are completely miscible in all proportions, whereas above the LCST, partial liquid miscibility occurs.

In some embodiments, adjunct 604 can include certain segments of a block copolymer that have an LCST ranging from approximately 20° C. to approximately 35° C. This allows at least a portion of the adjunct to undergo a phase transition at lower temperatures compared to a polyurethane backbone. Upon passing through the LCST, one of the polar or non-polar portions of the adjunct can micellize and form effective crosslinks within the system. In this manner, the fluid content increase due to fluid ingress can cause the adjunct to undergo a phase transition from one to two phases and then back to one phase. During the one phase, miscible period, the adjunct 604 has a low mechanical strength, which is desirable during deployment. During the two phase, immiscible period, fluid is absorbed into the adjunct 604 and the mechanical strength increases due to the effective increase in crosslinking that counterbalances the breakdown of the adjunct 604. Once a critical level is reached, the crosslinks can be disrupted, and the mechanical strength of the adjunct will decrease. This phase change allows for both low deployment forces and faster absorbing adjuncts since the critical phase of the healing process has reversible crosslinks.

In some embodiments, the second polymerizable compound can be a poloxamer added into the backbone of the polyurethane. Poloxamers are a class of water-soluble nonionic triblock copolymers formed by polar (polyethylene oxide) and non-polar (polypropylene oxide) blocks. Poloxamers can result in amphiphilic and surface-active properties to the polymer backbone. In some embodiments, increasing the water ingress into the polar portion of the adjunct 604 can promote faster absorption kinetics. The LCST is dependent on the polypropylene oxide segment length and relative water content.

In any of the embodiments described herein, the second polymerizable compound can be added as pendant groups to the first polymerizable compound backbone such that the second polymerizable compound forms a coating. The coating can act to prevent or delay the fluid ingress to the first polymerizable compound.

In some embodiments, the second polymerizable compound can act as an enteric coating in the presence of gastric fluid, or near an infection. The coating can be resistant or substantially resistant to absorption at certain pH, such as, for example, acidic conditions (pH<7) such that the coating can be used for tissue at or near the stomach. In other examples, the coating can delay absorption time for a predetermined timeframe and prevent the bulk material of the adjunct from contacting fluids. Gastric and infected fluids tend to have a lower pH than other locations in the body. Using an adjunct 604 with a second polymerizable compound that is resistant to degradation under acidic conditions can be beneficial when treating tissue near stomach, gastrointestinal tract, or other acidic areas of the body. In general, the retention of mechanical strength of the adjunct 604 is a function of absorption and fluid ingress. The second polymerizable compound can prevent or slow fluid penetration at lower pH's. As the pH increases, fluid is allowed to pass through the coating and initiate the degradation of the first polymerizable compound. The second polymerizable compound can be tailored to have a specific induction period based on pH by adjusting the composition, thickness, branching, and the like.

In some embodiments, the second polymerizable compound can be a polymethacrylate based copolymer with specific carboxylic acid functionalization. In addition, the second polymerizable compound can also include as a copolymer with or without the polymethacrylate, without limitation, a polydioxanone, a polyanhydride, a hydroxypropyl methylcellulose acetate succinate, a cellulose acetate phthalate, a cellulose acetate trimellitate, a hydroxypropyl methylcellulose phthalate, a polyvinyl acetate phthalate, a poly(trimethylene carbonate), a poly(beta-thioether ester ketal), a polypropylene fumarate, a poly(ester urea), a poly(ester amide), or combinations thereof.

In addition to a single enteric coating layer, the second polymerizable compound also forms a multilayer construct to provide additional advantages independent of the local pH environment. In some embodiments, the second polymerizable compound can form an acid-generating surface of the adjunct such that the enteric coating is maintained as additional protons or acid species are generated. This would extend the induction period provided by the enteric coating and further prevent any fluid ingress. As the surface eroding polymer degrades to a critical point, such as when the enteric coating is in the presence of a neutral pH, fluid ingress may proceed and allow for stapling implant absorption. In some embodiments, a multilayer construct may exist as a bilayer. In any embodiment described herein, the bilayer or multilayer construct can include active pharmaceutical ingredients (API's) that could be released as the surface eroding polymer degrades. Example acid-generating polymers can include, without limitation, polyanhydrides such as poly(carboxyphenoxy hexane-sebacic acid), poly(fumaric acid-sebacic acid), poly(imide-sebacic acid), and poly(imide-carboxyphenoxy hexane). Other acid-generating polymers can include 3-iodopropyl acetal moieties.

In some embodiments, when the second polymerizable compound forms a coating, the composition and/or thickness of the coating can provide an induction period for the absorption and/or degradation process. The initial thickness of the second polymerizable compound coating prior to delivery and/or an induction period can range from about 20 μm to about 100 μm (e.g., from about 30 μm to about 90 μm, from about 40 μm to about 60 μm, from about 45 μm to about 50 μm, and any range in between). Upon exposure to a temperature or a pH, the thickness of the second polymerizable compound coating may increase depending on the compression of the tissue required to reach a hemostatic seal.

As would be appreciated by one of skill in the art, the addition of the second polymerizable compound can be done with spatial control for optimal absorption, tissue in-growth, and/or hemostatic behavior. Further, the composition of the first and second polymerizable compound can be a gradient along a portion of the adjunct 604 (e.g., along a length, at a central point, at the ends, along the staple line, etc.,) with a varying bio-absorption profile. In general, a short-term absorption profile may be preferred to address hemostasis while a long-term absorption profile may address better tissue healing without leakages.

In some embodiments, adding the second polymerizable compound to the backbone, as pendant groups to the first polymerizable compound, or as a coating, can be done through any suitable technique including, without limitation, inkjet printing, direct deposition, thermal spraying, cold dynamic spraying, cold spraying, electro spraying, ultrasonic spray coating, dip coating, screen printing, spin coating, solution deposition, stereolithography, exterior lamination, and the like.

In some embodiments, the change in crosslink density between the first polymerizable compound and the second polymerizable compound forms a gradient of compression strength along a portion of the adjunct 604. In general, the adjunct 604 may have a compression strength of about 20 kPa to about 70 kPa, such as about 30 kPa to about 60 kPa (e.g., about 42 kPa), about 30 kPa to about 50 kPa, about 32.5 kPa to about 37.5 kPa. In some embodiments, the adjunct 604 may have a compression strength of about 20 kPa to about 70 kPa due to a portion of the adjunct 604 having the first polymerizable compound, and a compression strength of about 15 kPa to about 50 kPa due to a portion of the adjunct 604 having the second polymerizable compound.

In some examples, the adjunct 604 may have a tensile strength of about 30 kPa to about 90 kPa such as about 45 kPa to about 85 kPa or about 55 kPa to about 75 kPa. In some examples, the adjunct 604 will have tensile strength of about 110 kPa to about 150 kPa.

In any of the embodiments disclosed herein, the adjunct having a block copolymer having the first polymerizable compound and the second polymerizable compound can modulate the degradation profile of the adjunct (and the mechanical properties of the bioabsorbable material) for a predetermined time frame. For instance, a phase change may occur over several seconds or minutes such that during delivery, the porous body is readily compressible, and the end effector is easily inserted through the trocar. After the induction period passes, the mechanical strength of the adjunct 604 increases to allow for a suitable hemostatic seal between the tissue. This increased mechanical strength after the induction period can range from approximately 10 minutes to approximately 6 weeks, the amount of time that may be necessary for the tissue to heal.

Turning back to FIG. 9A, the end effector 106 is shown in a delivery configuration, where the adjunct 604 is compressed between the anvil 102 and the cartridge 200. As illustrated, the end effector 106 can include a staple cartridge 200 and an adjunct 604 comprising a bioabsorbable material. In certain embodiments, the adjunct 604 is releasably retained on the cartridge 200. When the end effector 106 closes around tissue T, the adjunct 604 can compress from a thickness of the uncompressed adjunct UT to a thickness of compressed adjunct CT of adjunct in response to a variation in tissue thickness.

FIG. 9B is a side view of the end effector 106 after firing of staples 300, where the adjunct 604 is no longer compressed between the anvil 102 and the cartridge 200. As shown, the adjunct 604 is released from the cartridge 200 after firing. Adjunct 604 is shown to maintain the thickness of the compressed adjunct CT and the thickness of uncompressed adjunct UT from pre-firing in FIG. 9A. In some embodiments, the adjunct 604 can undergo a phase change before firing of staples such that the adjunct height UT and compressed portion CT will be maintained after firing and releasing adjunct 604 and tissue T. In other embodiments, the adjunct 604 can undergo phase changes after a predetermined timeframe, as described above. In such an example, the adjunct height UT and compressed portion CT can either expand or contract in response to the staple height and the tissue thickness. As would be appreciated by those of skill in the art, a phase change of the adjunct 604 upon exposure to fluid ingress can be advantageous to ensure a hemostatic seal during changes in tissue inflammatory responses and throughout the healing process.

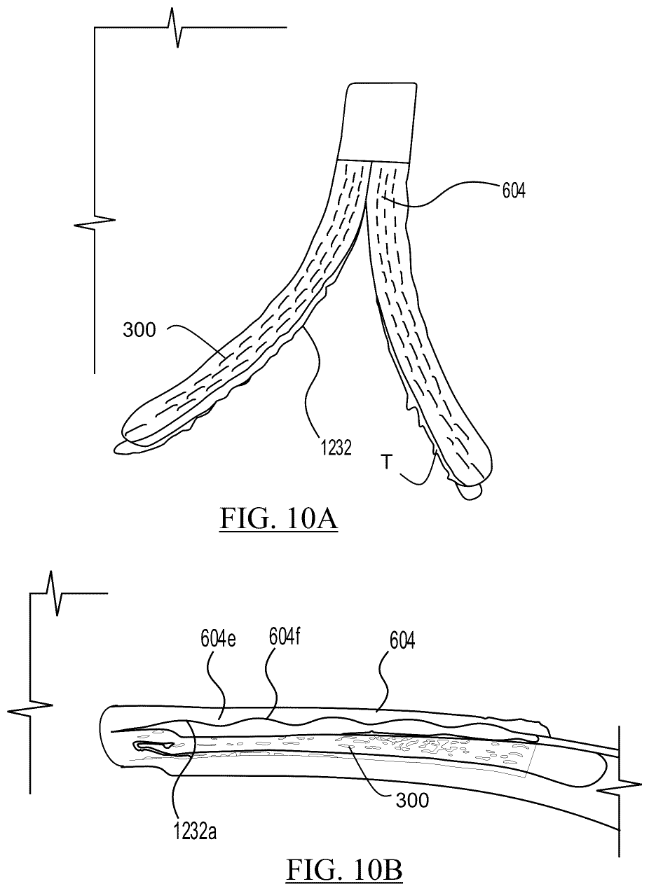

FIGS. 10A and 10B show top and side view of an adjunct 604 after firing staples 300 and cutting tissue T. Adjunct 604 may split in two post firing and the adhesive 1232 adhering the adjunct 604 to the cartridge 200 (see FIGS. 6A and 6B) may with the adjunct 604 form bumps 604e, 604f in the adjunct and corresponding bumps 1232a. Where the adjunct 604 contacts with the tissue T, bumps 604e, 604f may form that correspond to the texture and thickness variation in the tissue. This means that adjunct 604 can adapt to different heights and compressions depending on the application.

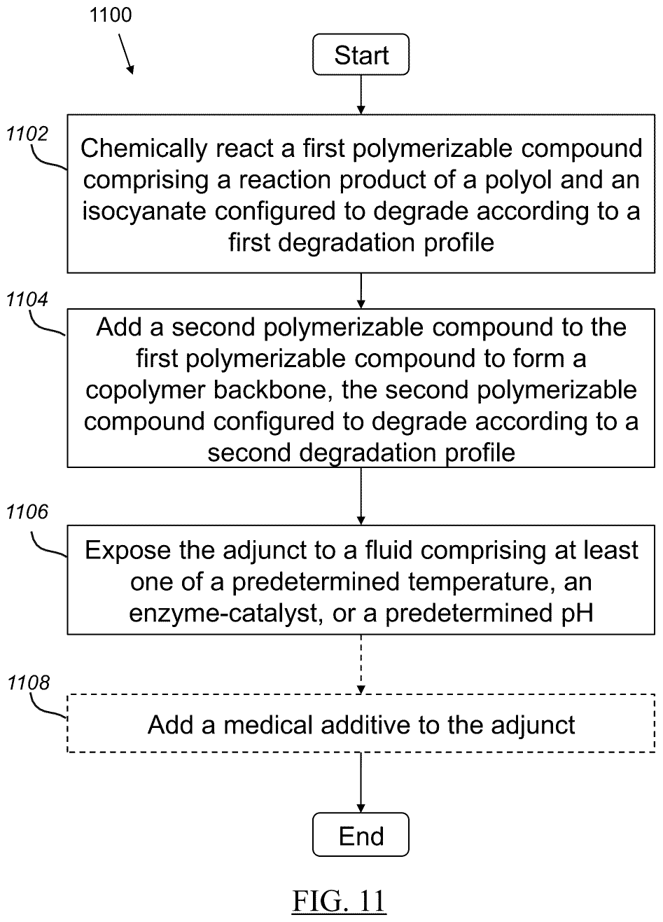

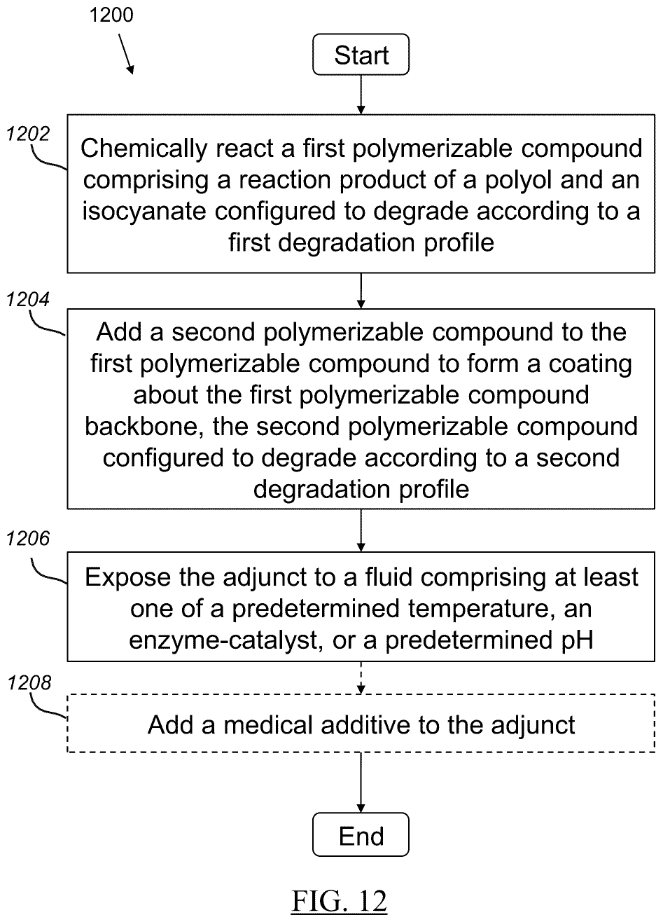

FIGS. 11 and 12 are flowcharts of methods 1100 and 1200 of forming the surgical adjunct 604, including a bioabsorbable material. The techniques for tuning the absorption pathway and change mechanical properties described herein may offer an added benefit of increasing adjunct strength and durability in combination with positive in vivo interactions (e.g., biocompatibility, wound healing, tissue integration, chemotherapy, anti-inflammatory, bone growth and integration, ligament and tendon repair, etc.) when the adjunct is delivered to the tissue site, as described herein. As such, the techniques described herein may allow the bioabsorbable material itself to aid in the healing process of the surrounding tissue. In addition, the embedding techniques described herein may offer an added benefit of preventing fibrous encapsulation of the foam cushion, and/or providing tunable release profiles for a variety of medical additives delivered to the tissue site.

Specifically with respect to FIG. 11, method 1100 used for tuning the absorption pathways in a bioabsorbable material (e.g., a foam) may include chemically reacting a first polymerizable compound comprising a reaction product of a polyol and an isocyanate configured to degrade according to a first degradation profile (step 1102). In some examples, the first polymerizable compound may include a polyurethane, a polyester urethane, a polyester urea, a polyester, a polycarbonate, a polyorthoester, a polyanhydride, a polyesteramide, a polyphosphazenes, a polyphosphoesters, a polysaccharides, and/or a polyoxaester. Method 1100 also includes adding a second polymerizable compound to the first polymerizable compound to form a copolymer backbone with the first polymerizable compound. (step 1104). The second polymerizable compound is configured to degrade according to a second degradation profile. After introduction of the second polymerizable compound, method 1100 includes exposing the adjunct to a fluid comprising at least one of a predetermined temperature (higher or lower), an enzyme-catalyst, or a predetermined pH (step 1106). Method 1100 can end after step 1106 or can optionally include adding a medical additive to the porous body (optional step 1108). In such examples, the medical additives may include medicants to treat pain and/or promote wound healing, tissue growth, infection reduction, and the like.

FIG. 12, similar to FIG. 11, includes a method 1200 used for tuning the absorption pathways in a bioabsorbable material (e.g., a foam), but instead of forming a backbone including both the first polymerizable compound and the second polymerizable compound of step 1104, method 1200 includes adding the second polymerizable compound to the first polymerizable compound to form a coating about the backbone. (step 1204). The coating can be resistant or substantially resistant to absorption at certain pH, such as, for example, acidic conditions (pH<7) such that the coating can be used for tissue at or near the stomach. In other examples, the coating can delay absorption time for a predetermined timeframe and prevent the bulk material of the adjunct from contacting fluids. The first polymerizable compound can form the backbone of the bioabsorbable material. As described above, the first polymerizable compound may include a polyurethane, a polyester urethane, a polyester urea, a polyester, a polycarbonate, a polyorthoester, a polyanhydride, a polyesteramide, a polyphosphazenes, a polyphosphoesters, a polysaccharides, and/or a polyoxaester. The second polymerizable compound is configured to degrade according to a second degradation profile. After introduction of the second polymerizable compound, method 1200 includes exposing the adjunct to a fluid comprising at least one of a predetermined temperature, an enzyme-catalyst, or a predetermined pH (step 1206). The second polymerizable compound can prevent or slow fluid penetration at lower pH's. As the pH increases, fluid is allowed to pass through the coating and initiate the degradation of the first polymerizable compound. Method 1200 can end after step 1206 or can optionally include adding a medical additive to the porous body (optional step 1208).

As will be appreciated by one skilled in the art, The embodiments described above are cited by way of example, and the present invention is not limited by what has been particularly shown and described hereinabove. Rather, the scope of the invention includes both combinations and sub combinations of the various features described hereinabove, as well as variations and modifications thereof which would occur to persons skilled in the art upon reading the foregoing description and which are not disclosed in the prior art.

In some examples, disclosed devices (e.g., end effector, surgical adjunct, and/or staple cartridges) and methods involving one or more disclosed devices may involve one or more of the following clauses:
Clause 1: A bioabsorbable material configured to be delivered to tissue, the material comprising: a first polymerizable compound comprising a reaction product of a polyol and an isocyanate and configured to degrade according to a first degradation profile, and a second polymerizable compound configured to degrade according to a second degradation profile different than the first degradation profile; wherein the material undergoes a phase transition in response to exposure to a fluid comprising at least one of a predetermined temperature, an enzyme-catalyst, and a predetermined pH.
Clause 2: The material of clause 1, wherein the first and second polymerizable compounds together form a copolymer backbone.
Clause 3: The material of clause 2, wherein the second polymerizable compound comprises a polyester, a poloxamer, a polypropylene oxide, a polyvinyl pyrrolidone, a poly(l-lactic acid), a poly(lactic-co-glycolic acid), or combinations thereof.
Clause 4: The material of clause 2, wherein a volumetric ratio of the second polymerizable compound to first polymerizable compound is in a range of about 0.125 to about 0.325.
Clause 5: The material of clause 1, wherein the material undergoes phase transition from a swollen state to a collapsed state when exposed to a temperature above the predetermined temperature.
Clause 6: The material of clause 5, wherein the first polymerizable compound and the second polymerizable compound degrade according to the respective first and second degradation profiles when the fluid comprises at least one of a temperature above the predetermined temperature and a pH above the predetermined pH.
Clause 7: The material of clause 1, wherein the predetermined temperature of the phase transition ranges from about 25° C. to about 35° C.
Clause 8: The material of clause 1, wherein the second polymerizable compound forms a coating about the first polymerizable compound such that the coating prevents the first polymerizable compound from exposure to a fluid ingress for a predetermined period of time.
Clause 9: The material of clause 8, wherein the second polymerizable compound comprises a polymethacrylate, a polydioxanone, a polyanhydride, a hydroxypropyl methylcellulose acetate succinate, a cellulose acetate phthalate, a cellulose acetate trimellitate, a hydroxypropyl methylcellulose phthalate, a polyvinyl acetate phthalate, a poly(trimethylene carbonate), a poly(beta-thioether ester ketal), a polypropylene fumarate, a poly(ester urea), a poly(ester amide), or combinations thereof.
Clause 10: The material of clause 8, wherein the predetermined period of time ranges from approximately 10 minutes to approximately 6 weeks.
Clause 11: The material of clause 8, wherein the coating comprises a thickness of about 20 μm to about 100 μm.
Clause 12: The material of clause 8, wherein the predetermined pH ranges from about 6.5 to about 7.4.
Clause 13: The material of clause 1, wherein the material has a compression strength of about 20 to about 70 kPa.
Clause 14: The material of clause 1, further comprising one or more medical additives configured to remain chemically bonded to at least one of the first polymerizable compound or the second polymerizable compound.
Clause 15: The material of clause 14, wherein the one or more medical additives are further configured to be released into the tissue over a predetermined period of time during at least one of the first degradation profile and the second degradation profile.
Clause 16: A bioabsorbable material configured to be delivered to tissue, the material comprising: a copolymer backbone comprising: a first polymerizable compound comprising a reaction product of a polyol and an isocyanate and configured to degrade according to a first degradation profile, and a second polymerizable compound configured to degrade according to a second degradation profile different than the first degradation profile; wherein the material undergoes a phase transition in response to exposure to the fluid comprising a predetermined temperature.
Clause 17: The material of clause 16, wherein the first polymerizable compound and the second polymerizable compound degrade according to the respective first and second degradation profiles when the fluid comprises a temperature above the predetermined temperature.
Clause 18: A bioabsorbable material configured to be delivered to tissue, the material comprising: a first polymerizable compound comprising a reaction product of a polyol and an isocyanate and configured to degrade according to a first degradation profile, and a second polymerizable compound coating the first polymerizable such that the coating prevents the first polymerizable compound from exposure to a fluid ingress for a predetermined period of time; wherein the material undergoes a phase transition in response to exposure to a fluid comprising a predetermined pH.
Clause 19: The material of clause 18, wherein the predetermined period of time ranges from approximately 10 minutes to approximately 6 weeks and the predetermined pH ranges from about 6.5 to about 7.4.
Clause 20: The material of clause 18, wherein the coating comprises a thickness of about 20 μm to about 100 μm.
Clause 21: A bioabsorbable material configured to be delivered to tissue, the material comprising: a first polymerizable compound comprising a reaction product of a polyol and an isocyanate and configured to degrade according to a first degradation profile, and a second polymerizable compound configured to degrade according to a second degradation profile different than the first degradation profile; wherein the material undergoes a phase transition in response to exposure to a fluid comprising at least one of a predetermined temperature, an enzyme-catalyst, and a predetermined pH.
Clause 22: The material of clause 21, wherein the first and second polymerizable compounds together form a copolymer backbone.
Clause 23: The material of clauses 21-22, wherein the second polymerizable compound comprises a polyester, a poloxamer, a polypropylene oxide, a polyvinyl pyrrolidone, a poly(l-lactic acid), a poly(lactic-co-glycolic acid), or combinations thereof.
Clause 24: The material of any of clauses 21-23, wherein a volumetric ratio of the second polymerizable compound to first polymerizable compound is in a range of about 0.125 to about 0.325.
Clause 25: The material of any of clauses 21-24, wherein the material undergoes phase transition from a swollen state to a collapsed state when exposed to a temperature above the predetermined temperature.
Clause 26: The material of any of clauses 21-25, wherein the first polymerizable compound and the second polymerizable compound degrade according to the respective first and second degradation profiles when the fluid comprises one or more of a temperature above the predetermined temperature and a pH above the predetermined pH.
Clause 27: The material of any of clauses 21-26, wherein the predetermined temperature of the phase transition ranges from about 25° C. to about 35° C.
Clause 28: The material of clause 21, wherein the second polymerizable compound forms a coating about the first polymerizable compound such that the coating prevents the first polymerizable compound from exposure to a fluid ingress for a predetermined period of time.
Clause 29: The material of clause 28, wherein the second polymerizable compound comprises a polymethacrylate, a polydioxanone, a polyanhydride, a hydroxypropyl methylcellulose acetate succinate, a cellulose acetate phthalate, a cellulose acetate trimellitate, a hydroxypropyl methylcellulose phthalate, a polyvinyl acetate phthalate, a poly(trimethylene carbonate), a poly(beta-thioether ester ketal), a polypropylene fumarate, a poly(ester urea), a poly(ester amide), or combinations thereof.
Clause 30: The material of any of clauses 28-29, wherein the predetermined period of time ranges from approximately 10 minutes to approximately 6 weeks.
Clause 31: The material of any of clauses 28-30, wherein the second polymerizable compound comprises a thickness of about 20 μm to about 100 μm.
Clause 32: The material of any of clauses 28-31 wherein the predetermined pH ranges from about 6.5 to about 7.4.
Clause 33: The material of any of clauses 21-32, wherein the material has a compression strength of about 20 to about 70 kPa.
Clause 34: The material of any of clauses 21-33, further comprising one or more medical additives configured to remain chemically bonded to one or both of the first polymerizable compound and the second polymerizable compound.
Clause 35: The material of any of claims 21-34, wherein the one or more medical additives are further configured to be released into the tissue over a predetermined period of time during one or both of the first degradation profile and the second degradation profile.

1-15. (canceled)

16. A bioabsorbable material configured to be delivered to tissue, the material comprising: a first polymerizable compound comprising a reaction product of a polyol and an isocyanate and configured to degrade according to a first degradation profile, and a second polymerizable compound configured to degrade according to a second degradation profile different than the first degradation profile; wherein the material undergoes a phase transition in response to exposure to a fluid comprising at least one of a predetermined temperature, an enzyme-catalyst, and a predetermined pH.

17. The material of claim 16, wherein the first and second polymerizable compounds together form a copolymer backbone.

18. The material of claim 17, wherein the second polymerizable compound comprises a polyester, a poloxamer, a polypropylene oxide, a polyvinyl pyrrolidone, a poly(l-lactic acid), a poly(lactic-co-glycolic acid), or combinations thereof.

19. The material of claim 17, wherein a volumetric ratio of the second polymerizable compound to first polymerizable compound is in a range of about 0.125 to about 0.325.

20. The material of claim 16, wherein the material undergoes phase transition from a swollen state to a collapsed state when exposed to a temperature above the predetermined temperature.

21. The material of claim 20, wherein the first polymerizable compound and the second polymerizable compound degrade according to the respective first and second degradation profiles when the fluid comprises at least one of a temperature above the predetermined temperature and a pH above the predetermined pH.

22. The material of claim 16, wherein the predetermined temperature of the phase transition ranges from about 25° C. to about 35° C.

23. The material of claim 16, wherein the second polymerizable compound forms a coating about the first polymerizable compound such that the coating prevents the first polymerizable compound from exposure to a fluid ingress for a predetermined period of time.

24. The material of claim 23, wherein the second polymerizable compound comprises a polymethacrylate, a polydioxanone, a polyanhydride, a hydroxypropyl methylcellulose acetate succinate, a cellulose acetate phthalate, a cellulose acetate trimellitate, a hydroxypropyl methylcellulose phthalate, a polyvinyl acetate phthalate, a poly(trimethylene carbonate), a poly(beta-thioether ester ketal), a polypropylene fumarate, a poly(ester urea), a poly(ester amide), or combinations thereof.

25. The material of claim 23, wherein the predetermined period of time ranges from approximately 10 minutes to approximately 6 weeks.

26. The material of claim 23, wherein the coating comprises a thickness of about 20 μm to about 100 μm.

27. The material of claim 23, wherein the predetermined pH ranges from about 6.5 to about 7.4.

28. The material of claim 16, wherein the material has a compression strength of about 20 to about 70 kPa.

29. The material of claim 16, further comprising one or more medical additives configured to remain chemically bonded to at least one of the first polymerizable compound or the second polymerizable compound.

30. The material of claim 29, wherein the one or more medical additives are further configured to be released into the tissue over a predetermined period of time during at least one of the first degradation profile and the second degradation profile.

31. A bioabsorbable material configured to be delivered to tissue, the material comprising: a copolymer backbone comprising: a first polymerizable compound comprising a reaction product of a polyol and an isocyanate and configured to degrade according to a first degradation profile, and a second polymerizable compound configured to degrade according to a second degradation profile different than the first degradation profile; wherein the material undergoes a phase transition in response to exposure to the fluid comprising a predetermined temperature.

32. The material of claim 31, wherein the first polymerizable compound and the second polymerizable compound degrade according to the respective first and second degradation profiles when the fluid comprises a temperature above the predetermined temperature.

33. A bioabsorbable material configured to be delivered to tissue, the material comprising: a first polymerizable compound comprising a reaction product of a polyol and an isocyanate and configured to degrade according to a first degradation profile, and a second polymerizable compound coating the first polymerizable such that the coating prevents the first polymerizable compound from exposure to a fluid ingress for a predetermined period of time; wherein the material undergoes a phase transition in response to exposure to a fluid comprising a predetermined pH.

34. The material of claim 33, wherein the predetermined period of time ranges from approximately 10 minutes to approximately 6 weeks and the predetermined pH ranges from about 6.5 to about 7.4.

35. The material of claim 33, wherein the coating comprises a thickness of about 20 μm to about 100 μm.
